(19) United States
(12) Patent Application Publication
(10) Pub. No.: US 2025/0217665 A1
(43) Pub. Date: Jul. 3, 2025

(54) INTELLIGENT METHOD TO MINT BLOCKCHAIN TRANSACTION BASED ON INFORMATION IN DNA APPARATUS LEVERAGING GENERATIVE AI

(71) Applicant: Bank of America Corporation, Charlotte, NC (US)

(72) Inventor: Shailendra Singh, Maharashtra (IN)

(21) Appl. No.: 18/401,641

(22) Filed: Jan. 1, 2024

(51) Int. Cl. G06N 3/123; G06N 10/80

(52) U.S. Cl. CPC G06N 3/123; G06N 10/80

(57) ABSTRACT

A method, apparatus, and system for minting blockchain transactions, leveraging generative artificial intelligence (“AI”), based on information stored in a deoxyribonucleic acid (“DNA”) storage apparatus is provided. The method, apparatus, and system may include receiving a request to execute a transaction from a generative AI user prompt interface. The method, apparatus, and system may include, in response to receipt of the request, analyzing the request to identify user requirements associated with the request, generating a smart contract for each of the user requirements, and converting each of the user requirements to a binary code. The method, apparatus, and system may include synthesizing a DNA sequence including nucleotides, each group of the nucleotides corresponding to one of the binary codes. The method, apparatus, and system may include, after the synthesizing of the DNA sequence, producing a blockchain storing data relating to the transaction using data stored in the DNA sequence.

### FIELD OF TECHNOLOGY

Aspects of the disclosure relate to generative artificial intelligence (“AI”) and deoxyribonucleic acid (“DNA”)-based data storage for blockchain production.

### INCORPORATION BY REFERENCE OF A SEQUENCE LISTING XML

A Sequence Listing is provided herewith as a Sequence Listing XML, “104-1038XMLSequenceListing.xml” created on Feb. 26, 2024 and having a size of 2.03 KB. The contents of the Sequence Listing XML are incorporated by reference herein in their entirety.

### BACKGROUND OF THE DISCLOSURE

DNA storage is a digital data storage technology based on specific encoding and decoding methods. The methods provided may include encoding and decoding between binary codes of digital data (e.g., 0s and 1s) and adenosine-thymine-cytosine-guanine (“A-T-C-G”) quaternary nucleotide codes of DNA.

DNA storage is expected to develop into a major data storage form in the future due to its advantages. Advantages of DNA storage include high data density, long storage times, low energy consumption, convenience for carrying data, concealed transportation, and capabilities for multiple encryptions.

There exists a need to produce blockchains based on information stored in a DNA storage apparatus. It would be desirable, therefore, to develop systems and methods of minting blocks on a blockchain of transactions based on a user prompt for a financial institution request, while also leveraging the advantages of DNA storage.

### BRIEF DESCRIPTION OF THE DRAWINGS

The objects and advantages of the disclosure will be apparent upon consideration of the following detailed description, taken in conjunction with the accompanying drawings, in which like reference characters refer to like parts throughout, and in which:

FIG. 1 shows an illustrative system in accordance with the principles of the disclosure;

FIG. 2 shows an illustrative flow diagram in accordance with principles of the disclosure;

FIG. 3 shows an illustrative flow diagram in accordance with principles of the disclosure;

FIG. 4 shows an illustrative diagram in accordance with principles of the disclosure;

FIG. 5 shows an illustrative system in accordance with the principles of the disclosure;

FIG. 6 shows an illustrative system in accordance with the principles of the disclosure; and

FIG. 7 shows illustrative information in accordance with the principles of the disclosure along with some of the system shown in FIG. 6.

### DETAILED DESCRIPTION OF THE DISCLOSURE

A method, apparatus, and system are provided for synthesizing a DNA sequence. The method, apparatus, and system may produce a distributed blockchain of transactions based on information stored in the DNA sequence. The method, apparatus, and system may leverage AI and machine learning (“ML”). ML is a deep learning tool that can improve computer algorithms, searches, and computations. ML may work in tandem with AI, e.g., AI-ML. AI-ML may be a self-learning tool utilizing both AI and ML.

The method, apparatus, and system provided herein straddle the fields of DNA computing for data storage, blockchain, generative AI, ML, and quantum computing. The method, apparatus, and system may provide an intelligent technical procedure for generating a distributed ledger (i.e., blockchain) of transactions based on information stored in a DNA storage apparatus leveraging AI-ML.

The method, apparatus, and system may identify a genesis nucleotide (e.g., A-T-G-C) sequence in a DNA decoding synthesis apparatus. The method, apparatus, and system may initiate a block creation procedure based on a smart contract rule. A nucleotide sequence search may be enabled through a quantum Grover's search algorithm running on quantum hardware.

In quantum computing, Grover's search algorithm is a quantum algorithm for unstructured search. Grover's search algorithm may predict an input function that may produce a particular output value. The Grover's search algorithm works by applying a series of quantum operations to the input function, acting as a superposition of all possible searches. Therefore, Grover's search algorithm enables more rapid searchability than classical search algorithms.

A smart contract may have a predefined set of rules to mint blocks. The predefined set of rules may mint blocks once the predefined set of rules match a DNA sequence. Smart contract rules may be dynamically generated based on user prompts. User prompts (in the form of requirements) may be mapped to transaction contexts. The method, apparatus, and system may generate smart contracts associated with the transaction contexts.

A smart contract may have a predefined set of rules to extract information stored in a DNA computing environment. The smart contract rules may include a program to validate a nucleotide sequence. The smart contract rules may include a program to trigger a genesis block and subsequent blocks.

The method, apparatus, and system may mint each block based on a corresponding DNA nucleotide sequence. The DNA storage and synthesis apparatus may contain block initiation nucleotides for genesis blocks and block termination nucleotides for termination blocks.

The method, apparatus, and system may use a DNA synthesis engine, a genesis nucleotide sequence identifier engine, a smart contract, a block validation engine, a transaction orchestration engine, a generative AI engine, a prompt engine, and a quantum search engine. The quantum search engine may include a Grover's search algorithm.

The DNA synthesis engine may synthesize a DNA nucleotide sequence. The DNA synthesis engine may synthesize pools of DNA nucleotide sequences. A pool of DNA nucleotide sequences may include a plurality of DNA nucleotide sequences. The plurality of DNA nucleotide sequences may be searchable by the quantum search engine. The pools of DNA nucleotide sequences may be searchable by the quantum search engine.

The genesis nucleotide sequence identifier engine may identify a genesis nucleotide sequence in a DNA nucleotide sequence. The genesis nucleotide sequence may be the first nucleotide sequence that may mint a genesis block in a blockchain. The genesis block in a blockchain is the first block that triggers subsequent blocks minted in a blockchain.

The smart contract may contain a predefined set of rules to process information stored in DNA nucleotide sequences. Smart contracts may be stored in a repository of smart contracts. Smart contracts may extract information from DNA nucleotides sequences.

The block validation engine may validate each block in the blockchain. The block validation engine may ensure that each step in the blockchain is correct. The block validation engine may check whether the information obtained from the DNA sequence is accurate.

The transaction orchestration engine may control and maintain the blockchain. The transaction orchestration engine may publish the blockchain to a central server or network. The transaction orchestration engine may maintain the blockchain for a length of time. For example, the transaction orchestration engine may have a set of rules to maintain the blockchain for 1 week, 1 month, or 1 year.

The generative AI engine may use AI to generate, search, and convert DNA nucleotide sequences to computer language code and vice versa. The generative AI engine may use AI to store DNA nucleotide sequence information in a blockchain environment.

The prompt engine may prompt a user to enter information into a graphical user interface (“GUI”). The prompt engine may collect information from a user. For example, the prompt engine may about “know your customer” (“KYC”) information from a user. KYC information may include user ID, social security number, address, account balance, and tax information.

The quantum search engine may use Grover's search algorithms to search for information within DNA nucleotide sequences. The quantum search engine may enable a faster search for information needed for blockchain generation. DNA storage is usually slower than other storage methods because DNA synthesis is required. Thus, the quantum search engine may speed up the process of extracting information from a DNA nucleotide sequence. The quantum search engine may enable rapid block minting for a blockchain corresponding to a DNA nucleotide sequence.

A method for synthesizing a DNA sequence is provided. The method may include generating a distributed blockchain of transactions. The distributed blockchain of transactions may be based on information stored in the DNA sequence. The method may include leveraging AI. The method may include leveraging ML. The method may include leveraging a quantum search engine. The quantum search engine may utilize one or more of Grover's search algorithms. The quantum search engine may improve method efficiency.

The method may include receiving a request to execute a transaction from a generative AI user prompt interface. The generative AI user prompt interface may be a GUI.

The method may include, in response to receipt of the request, analyzing the request. The request may be analyzed to identify user requirements associated with the request. User requirements may be requirements to execute a transaction. For example, to execute a mortgage, user requirements may include, but are not limited to, username, password, address, personal identification number (“PIN”), credit score, fraud history, tax information, and telephone number.

The method may include generating a smart contract for each of the user requirements. The method may include converting each of the user requirements to a binary code.

The method may include synthesizing a DNA sequence including nucleotides, each group of the nucleotides corresponding to one of the binary codes. For example, adenosine (“A”) may correspond to “00,” thymine (“T”) may correspond to “01,” cytosine (“C”) may correspond to “01,” and guanine (“G”) may correspond to “11.” The synthesizing may encode the user requirements associated with the request.

The method may include, after the synthesizing of the DNA sequence, producing a blockchain. The blockchain may store data relating to the transaction. The blockchain may be produced using data stored in the DNA sequence.

The method may include extracting data stored in the DNA sequence. The method may include decoding the data extracted from the DNA sequence. The method may include identifying, in the decoded data, one of the user requirements.

The method may include, in response to identifying the user requirement, searching a database for a genesis nucleotide sequence. The genesis nucleotide sequence may correspond to the user requirement. The searching may be performed by AI-ML. The searching may be performed by generative AI. The searching may be performed by a quantum search engine.

The method may include, in response to identifying, in the database, the genesis nucleotide sequence, minting a genesis block. The genesis block may be stored in the genesis nucleotide sequence.

The method may include initiating the blockchain of transactions by storing the genesis block on the blockchain. The method may include minting a plurality of blocks to add to the blockchain. Each of the plurality of blocks may correspond to each of the user requirements associated with the request.

The method may include, after the minting of the genesis block and the plurality of blocks, adding the genesis block and the plurality of blocks to the blockchain. The method may include validating the blockchain using a blockchain validation engine.

The method may include searching the blockchain for a block termination nucleotide sequence via AI-ML. The method may include searching the blockchain for a block termination nucleotide sequence via generative AI. The method may include searching the blockchain for a block termination nucleotide sequence via a quantum search engine.

The method may include identifying one of the plurality of blocks as the block termination nucleotide sequence. The method may include finalizing the blockchain by converting the identified one of the plurality of blocks to a terminal block on the blockchain. The terminal block may store the block termination nucleotide sequence.

The method may include, after the extracting of the data stored in the DNA sequence, mapping the data extracted to a contextual data interface using AI-ML. The method may include mapping the contextual data interface further to the user requirements.

The method may include, after the minting of the genesis block, storing the genesis block on a network of computing systems. Each computing system may be a node in the blockchain.

The method may include, after the minting of the plurality of blocks, storing the plurality of blocks on the network of computing systems. The method may include using groups of the nucleotides in the DNA sequence. Each group of the nucleotides in the DNA sequence may correspond to each one of the binary codes includes A, T, C, and G. For example, A may correspond to “00,” T may correspond to “01,” C may correspond to “10,” and G may correspond to “11.”

The method may include using a quantum search engine. The quantum search engine may use Grover's search algorithm to search for data stored in the DNA sequence. The quantum search engine may enable faster searching of data required for the finalizing of the blockchain.

An apparatus for synthesizing a DNA sequence is provided. The apparatus may generate a distributed blockchain of transactions. The distributed blockchain of transactions may be based on information stored in the DNA sequence. The apparatus may leverage AI. The apparatus may leverage ML. The apparatus may leverage a quantum search engine.

The apparatus may include a computer processor. The apparatus may include a computer-readable medium. The computer-readable medium may be coupled with the computer processor. The computer-readable medium may store instructions. The computer processor may execute the instructions stored on the computer-readable medium. The instructions, when executed by the computer processor, may cause the computer processor to perform a method.

The apparatus may include a computer processor receiving a request to execute a transaction from a generative AI user prompt interface. The apparatus may include a computer processor receiving a request to execute a transaction from a generative AI user prompt interface. The generative AI user prompt interface may be a GUI.

The apparatus may include a computer processor, in response to receipt of the request, analyzing the request. The request may be analyzed to identify user requirements associated with the request. User requirements may be requirements to execute a transaction.

The apparatus may include a computer processor generating a smart contract for each of the user requirements. The apparatus may include a computer processor converting each of the user requirements to a binary code.

The apparatus may include a computer processor instructing a laboratory in synthesizing a DNA sequence including nucleotides. Each group of the nucleotides may correspond to one of the binary codes. For example, adenosine (“A”) may correspond to “00,” thymine (“T”) may correspond to “01,” cytosine (“C”) may correspond to “01,” and guanine (“G”) may correspond to “11.” The synthesizing may encode the user requirements associated with the request.

The apparatus may include a computer processor, after the synthesizing of the DNA sequence, producing a blockchain. The blockchain may store data relating to the transaction. The blockchain may be produced using data stored in the DNA sequence.

The apparatus may include a computer processor extracting data stored in the DNA sequence. The apparatus may include a computer processor decoding the data extracted from the DNA sequence. The apparatus may include a computer processor identifying, in the decoded data, one of the user requirements.

The apparatus may include a computer processor, in response to identifying the user requirement, searching a database for a genesis nucleotide sequence. The genesis nucleotide sequence may correspond to the user requirement. The searching may be performed by AI-ML. The searching may be performed by generative AI. The searching may be performed by a quantum search engine.

The apparatus may include a computer processor, in response to identifying, in the database, the genesis nucleotide sequence, minting a genesis block. The genesis block may be stored in the genesis nucleotide sequence.

The apparatus may include a computer processor initiating the blockchain of transactions by storing the genesis block on the blockchain. The apparatus may include a computer processor minting a plurality of blocks to add to the blockchain. Each of the plurality of blocks may correspond to each of the user requirements associated with the request.

The apparatus may include a computer processor, after the minting of the genesis block and the plurality of blocks, adding the genesis block and the plurality of blocks to the blockchain. The apparatus may include a computer processor validating the blockchain using a blockchain validation engine.

The apparatus may include a computer processor searching the blockchain for a block termination nucleotide sequence via AI-ML. The apparatus may include a computer processor searching the blockchain for a block termination nucleotide sequence via generative AI. The apparatus may include a computer processor the blockchain for a block termination nucleotide sequence via a quantum search engine.

The apparatus may include a computer processor identifying one of the plurality of blocks as the block termination nucleotide sequence. The apparatus may include a computer processor finalizing the blockchain by converting the identified one of the plurality of blocks to a terminal block on the blockchain. The terminal block may store the block termination nucleotide sequence.

The apparatus may include a computer processor, after the extracting of the data stored in the DNA sequence, mapping the data extracted to a contextual data interface using AI-ML. The apparatus may include a computer processor mapping the contextual data interface further to the user requirements.

The apparatus may include a computer processor, after the minting of the genesis block, storing the genesis block on a network of computing systems. Each computing system may be a node in the blockchain.

The apparatus may include a computer processor, after the minting of the plurality of blocks, storing the plurality of blocks on the network of computing systems. The apparatus may include a computer processor using groups of the nucleotides in the DNA sequence. Each group of the nucleotides in the DNA sequence may correspond to each one of the binary codes includes A, T, C, and G. For example, A may correspond to “00,” T may correspond to “01,” C may correspond to “10,” and G may correspond to “11.”

The apparatus may include a computer processor using a quantum search engine. The quantum search engine may use Grover's search algorithm to search for data stored in the DNA sequence. The quantum search engine may enable faster searching of data required for the finalizing of the blockchain.

A system for synthesizing a DNA sequence is provided. The system may generate a distributed blockchain of transactions. The distributed blockchain of transactions may be based on information stored in the DNA sequence. The system may leverage AI. The system may leverage ML. The system may leverage a quantum search engine.

The system may include a computer processor. The system may include a computer-readable medium. The computer-readable medium may be coupled with the computer processor. The computer-readable medium may store instructions. The computer processor may execute the instructions stored on the computer-readable medium. The instructions, when executed by the computer processor, may cause the computer processor to perform a method.

The system may include a computer processor receiving a request to execute a transaction from a generative AI user prompt interface. The system may include a computer processor receiving a request to execute a transaction from a generative AI user prompt interface. The generative AI user prompt interface may be a GUI.

The system may include a computer processor, in response to receipt of the request, analyzing the request. The request may be analyzed to identify user requirements associated with the request. User requirements may be requirements to execute a transaction.

The system may include a computer processor generating a smart contract for each of the user requirements. The system may include a computer processor converting each of the user requirements to a binary code.

The system may include a computer processor instructing a laboratory in synthesizing a DNA sequence including nucleotides. Each group of the nucleotides may correspond to one of the binary codes. For example, adenosine (“A”) may correspond to “00,” thymine (“T”) may correspond to “01,” cytosine (“C”) may correspond to “01,” and guanine (“G”) may correspond to “11.” The synthesizing may encode the user requirements associated with the request.

The system may include a computer processor, after the synthesizing of the DNA sequence, producing a blockchain. The blockchain may store data relating to the transaction. The blockchain may be produced using data stored in the DNA sequence.

The system may include a computer processor extracting data stored in the DNA sequence. The system may include a computer processor decoding the data extracted from the DNA sequence. The system may include a computer processor identifying, in the decoded data, one of the user requirements.

The system may include a computer processor, in response to identifying the user requirement, searching a database for a genesis nucleotide sequence. The genesis nucleotide sequence may correspond to the user requirement. The searching may be performed by AI-ML. The searching may be performed by generative AI. The searching may be performed by a quantum search engine.

The system may include a computer processor, in response to identifying, in the database, the genesis nucleotide sequence, minting a genesis block. The genesis block may be stored in the genesis nucleotide sequence.

The system may include a computer processor initiating the blockchain of transactions by storing the genesis block on the blockchain. The system may include a computer processor minting a plurality of blocks to add to the blockchain. Each of the plurality of blocks may correspond to each of the user requirements associated with the request.

The system may include a computer processor, after the minting of the genesis block and the plurality of blocks, adding the genesis block and the plurality of blocks to the blockchain. The system may include a computer processor validating the blockchain using a blockchain validation engine.

The system may include a computer processor searching the blockchain for a block termination nucleotide sequence via AI-ML. The system may include a computer processor searching the blockchain for a block termination nucleotide sequence via generative AI. The system may include a computer processor the blockchain for a block termination nucleotide sequence via a quantum search engine.

The system may include a computer processor identifying one of the plurality of blocks as the block termination nucleotide sequence. The system may include a computer processor finalizing the blockchain by converting the identified one of the plurality of blocks to a terminal block on the blockchain. The terminal block may store the block termination nucleotide sequence.

The system may include a computer processor, after the extracting of the data stored in the DNA sequence, mapping the data extracted to a contextual data interface using AI-ML. The system may include a computer processor mapping the contextual data interface further to the user requirements.

The system may include a computer processor, after the minting of the genesis block, storing the genesis block on a network of computing systems. Each computing system may be a node in the blockchain.

The system may include a computer processor, after the minting of the plurality of blocks, storing the plurality of blocks on the network of computing systems. The apparatus may include a computer processor using groups of the nucleotides in the DNA sequence. Each group of the nucleotides in the DNA sequence may correspond to each one of the binary codes includes A, T, C, and G. For example, A may correspond to “00,” T may correspond to “01,” C may correspond to “10,” and G may correspond to “11.”

The system may include a computer processor using a quantum search engine. The quantum search engine may use Grover's search algorithm to search for data stored in the DNA sequence. The quantum search engine may enable faster searching of data required for the finalizing of the blockchain.

Systems and methods in accordance with this disclosure will now be described in connection with the figures, which form a part hereof. The figures show illustrative features of system and method steps in accordance with the principles of this disclosure. It is to be understood that other embodiments may be utilized, and that structural, functional, and procedural modifications may be made without departing from the scope and spirit of the present disclosure.

The steps of methods may be performed in an order other than the order shown and/or described herein. Method embodiments may omit steps shown and/or described in connection with illustrative methods. Method embodiments may include steps that are neither shown nor described in connection with illustrative methods. Illustrative method steps may be combined. For example, an illustrative method may include steps shown in connection with any other illustrative method.

The systems and methods may omit features shown and/or described in connection with illustrative systems. System and method embodiments may include features that are neither shown nor described in connection with illustrative systems and methods. Features of illustrative systems and methods may be combined. For example, an illustrative system and method may include features shown or described in connection with another illustrative system/method.

As will be appreciated by one of skill in the art, the disclosure described herein may be embodied in whole or in part as a method, a data processing system, or a computer program product. Accordingly, the disclosure may take the form of entirely hardware, entirely software, or combining software, hardware and any other suitable approach or system.

Furthermore, such aspects may take the form of a computer program product stored by one or more computer-readable storage encoded media having computer-readable program code, or instructions, embodied in or on the storage encoded media. Any suitable computer readable storage encoded media may be utilized, including hard disks, CD-ROMs, optical storage devices, magnetic storage devices, and/or any combination thereof. In addition, various signals representing data or events as described herein may be transferred between a source and a destination in the form of electromagnetic waves traveling through signal-conducting encoded media such as metal wires, optical fibers, and/or wireless transmission encoded media (e.g., air and/or space

Illustrative information that is exchanged with the system may be transmitted and displayed using any suitable markup language under any suitable protocol, such as those based on JAVA, COCOA, XML, or any other suitable languages or protocols.

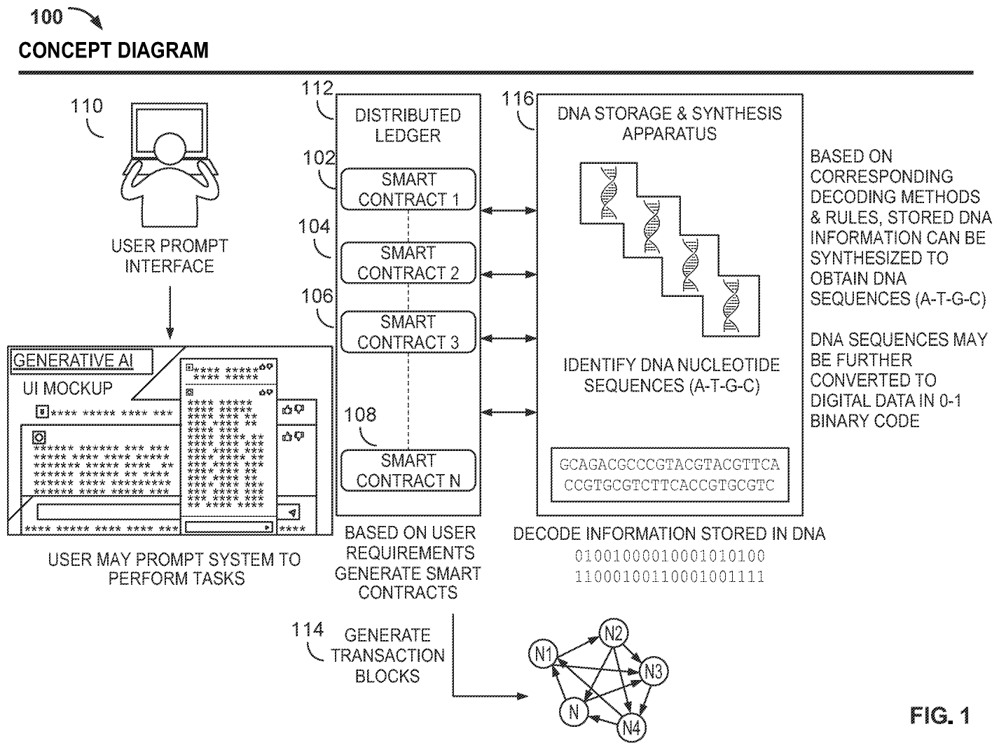

Processes in accordance with the principles of the disclosure may include one or more features of the processes illustrated in FIGS. 1-7. For the sake of illustration, the steps of the processes illustrated in FIGS. 1-7 will be described as performed by a “system.” The “system” may include one or more of the features of the systems that are shown or described herein and/or any other suitable device or approach. The “system” may be provided by an entity. The entity may be an individual, an organization or any other suitable entity.

Systems and methods described herein are illustrative. Systems and methods in accordance with this disclosure will now be described in connection with the figures, which form a part hereof. The figures show illustrative features of system and method steps in accordance with the principles of this disclosure. It is understood that other embodiments may be utilized, and that structural, functional, and procedural modifications may be made without departing from the scope and spirit of the present disclosure.

FIG. 1 shows an illustrative concept diagram 100 of synthesizing a DNA sequence and producing a distributed blockchain of transactions based on information stored in the DNA sequence leveraging AI and ML. In this illustrative diagram a user prompt interface 110 may leverage AI and ML to analyze transaction requests for user requirements.

Smart contract 1, at 102, may correspond to a first user requirement. Smart contract 1, at 102, may generate transaction blocks 114. The transaction blocks may be put on a blockchain. N may represent an initial transaction block producing a node on the blockchain. N1, N2, N3, and N4 may be subsequent transaction blocks and nodes on the blockchain. The blockchain nodes may all be in network communication.

Smart contract 1, at 102, may correspond to a first DNA nucleotide sequence. The first DNA nucleotide sequence may be synthesized by a DNA storage and synthesis apparatus 116.

Smart contract 2, at 104, may correspond to a second user requirement. Smart contract 2, at 104, may correspond to a second DNA nucleotide sequence. The second DNA nucleotide sequence may be synthesized by the DNA storage and synthesis apparatus 116. Smart contract 2, at 104, may generate transaction blocks 114.

Smart contract 3, at 106, may correspond to a third user requirement. And smart contract N, at 108, may correspond to an nth user requirement. Smart contract 3, at 106, may correspond to a third DNA nucleotide sequence. Smart contract 3, at 106, may generate transaction blocks 114.

Smart contract N, at 108, may correspond to an Nth DNA nucleotide sequence. N may represent a total number of DNA nucleotide sequences. Smart contract N, at 108, may generate transaction blocks 114.

Smart contracts 1 through N may include a distributed ledger 112. Distributed ledger 112 may include a blockchain.

The first through nth DNA nucleotide sequences may be synthesized by the DNA storage and synthesis apparatus 116. The first through Nth DNA nucleotide sequences may be single strands of DNA. The first through Nth DNA nucleotide sequences may be pools of DNA strands. The first through Nth DNA nucleotide sequences may exist in single strand or double helix configurations.

Based on corresponding decoding methods and rules, stored DNA information may be sequenced to obtain DNA sequences in A-T-G-C format. The DNA sequences in A-T-G-C format may be further converted to digital data in the form of 0s and 1s, i.e., binary code. For example, a DNA nucleotide sequence of “GCAGACGCCCGTACGTACGTTCACCGTGCGTCTTCACCGTGCGTC” may store user requirement information. The DNA nucleotide sequence may be decoded into binary code. The DNA sequence may correspond to binary code: “0100100001000101010011000100110001001111.” The binary code may also contain user requirement information.

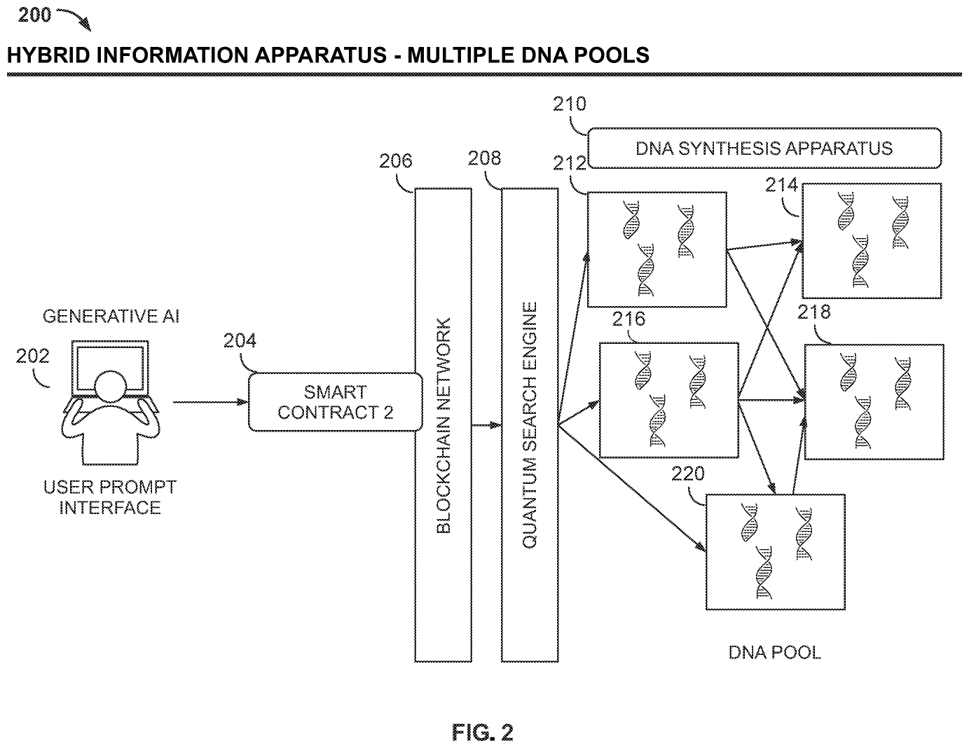

FIG. 2 shows an illustrative flow diagram representing a hybrid information apparatus producing multiple DNA pools 200. Hybrid information refers to both DNA sequence information and binary code information. The technical process steps of the flow diagram may include the following items.

Generative AI user prompt interface 202 may include a computer processor. Generative AI user prompt interface 202 may produce one or more smart contracts, e.g., smart contract 2, at 204.

Smart contract 2, at 204 may contain a predefined set of rules. The predefined set of rules may, when run by a computer processor, mint a block on a blockchain network, 206. Blockchain network 206 may be analyzed by a quantum search engine 208. Quantum search engine 208 may use Grover's search algorithm. Based on results from the quantum search engine 208, one or more DNA nucleotide sequences may be synthesized in a DNA synthesis apparatus 210. The DNA synthesis apparatus 210 may produce one or more DNA nucleotide sequences.

DNA nucleotide sequences may be grouped into clusters. Each cluster of DNA nucleotide sequences may represent one or more user requirements. Clusters of DNA nucleotides sequences may represent connected blockchain nodes. For example, cluster 1, 212, may represent one node in the blockchain. DNA cluster 1, 212, may be connected to DNA cluster 2, 214, which may represent a second node in the blockchain. And DNA cluster 3, 216, may be connected to DNA cluster 4, 218 and DNA cluster 5, 220, each a node in the blockchain. DNA clusters 1-5 may represent a DNA pool making up a blockchain.

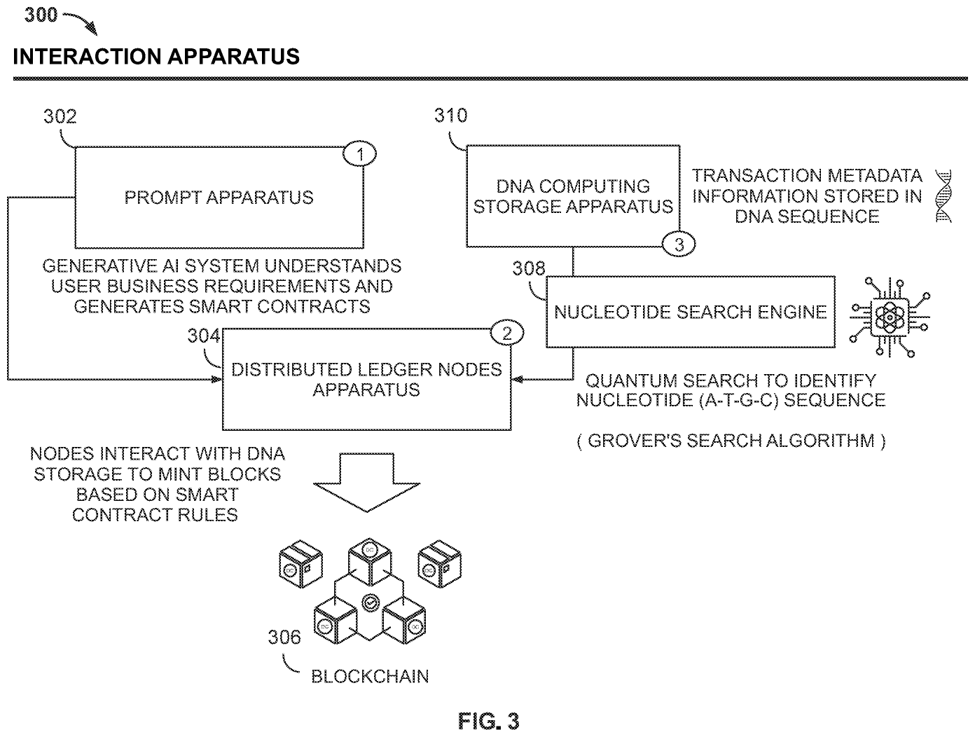

FIG. 3 shows an illustrative flowchart representing the interaction apparatus 300 of the disclosure.

Prompt apparatus 302, at 1, may represent a generative AI system that understands user requirements for transactions. At prompt apparatus 302, at 1, a user may be prompted to enter one or more transaction requests. Generative AI in prompt apparatus 302 may extract user requirements from the transaction requests. Prompt apparatus 302 may generate one or more smart contracts based on user requirements extracted from the transaction requests.

Distributed ledger nodes apparatus 304, at 2, may produce nodes for blockchain production. Nodes may interact with a DNA computing storage apparatus 310 to mint blocks. The blocks may be minted based on smart contract rules. The blocks may be put on a blockchain, e.g., blockchain 306.

Nucleotide search engine 308 may include a quantum search engine. The nucleotide search engine 308 may run a quantum search to identify a nucleotide (A-T-G-C) sequence. The nucleotide search engine 308 may run a Grover's search algorithm. The results of the nucleotide search engine 308 may be fed into distributed ledger nodes apparatus 304. The results of the nucleotide search engine 308 may mint blocks and produce nodes on the blockchain for distributed ledger nodes apparatus 304.

DNA computing storage apparatus 310, at 3, may extract transaction metadata information stored in a DNA nucleotide sequence. Quantum search engine 308 may run a Grover's search algorithm on a DNA sequence in the DNA computing storage apparatus 310.

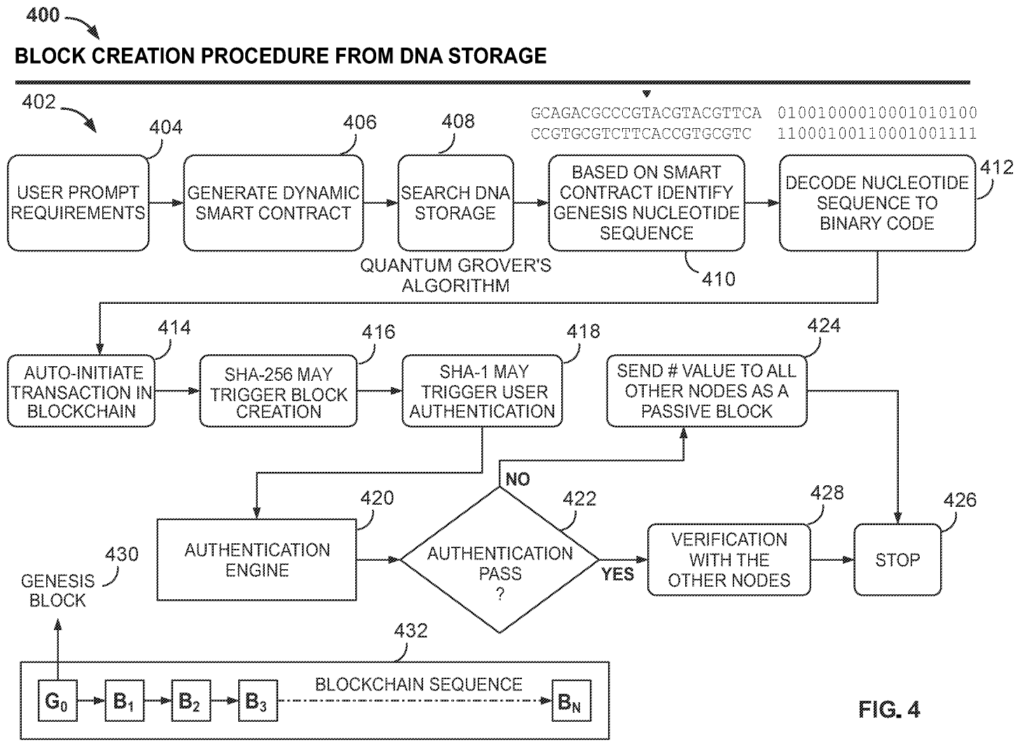

FIG. 4 shows an illustrative flow diagram 402 representing an exemplary block creation procedure from DNA storage 400.

At user prompt requirements 404, a user may enter a request into a GUI. Generative AI in user prompt requirements 404 may extract user requirements from the user request. User prompt requirements 404 may be connected to generate dynamic smart contract 406.

At generate dynamic smart contract 406, a dynamic smart contract may be generated based on the user requirements extracted from generative AI. A dynamic smart contract may be an alterable smart contract. A dynamic smart contract may contain a predefined set of instructions to mint blocks. Generate dynamic smart contract 406 may be connected to search DNA storage 408.

At search DNA storage 408, DNA information stored in a computer processor may be searched, analyzed, and extracted. Search DNA storage 408 may be performed using a quantum Grover's search algorithm. Search DNA storage 408 may be connected to the next step-based on smart contract, identify a genesis nucleotide sequence 410.

At based on smart contract, identify a genesis nucleotide sequence 410, a smart contract containing a predefined set of rules for a genesis nucleotide sequence 410 may be utilized. Information regarding the genesis nucleotide sequence 410 may be extracted from the smart contract.

The genesis nucleotide sequence 410 may include a unique DNA nucleotide sequence, e.g., “GCAGACGCCCGTACGTACGTTCACCGTGCGTCTTCACCGTGCGTC.” Based on smart contract, identify a genesis nucleotide sequence 410 may be connected to the next step—decode the nucleotide sequence into binary code 412.

At decode the nucleotide sequence into binary code 412, a DNA nucleotide sequence may be converted into 0s and 1s in binary code. The binary code may include, e.g., “0100100001000101010011000100110001001111.” Decode the nucleotide sequence into binary code 412 may be connected to the next step—auto-initiate a transaction in a blockchain 414. At auto-initiate a transaction in a blockchain 414, a blockchain is formed from blocks minted from smart contracts.

In cryptography, Secure Hash Algorithm (“SHA”)-1 is a hash function that may take an input and may produce a 160-bit (20-byte) hash value (e.g., a message digest), usually rendered as 40 hexadecimal digits. SHA-2 changed from its predecessor, SHA-1. The SHA-2 family consists of six hash functions with digests (hash values) that are 224, 256, 384 or 512 bits. For example, SHA-256 may generate a 256-bit (32-byte) hash. A hash may include a one-way function. A hash may be suitable for checking the integrity of data, challenge hash authentications, anti-tamper functions, digital signatures, and blockchains.

Auto-initiate a transaction in a blockchain 414 may be connected to the next step, SHA-256 may trigger block creation 416. At SHA-256 may trigger block creation 416, block creation may be triggered by a genesis block 430, e.g., G₀. The genesis block 430, G₀, may then lead to a blockchain sequence 432, e.g., B₁, B₂, B₃, and B_{N}. N may represent a total number of blocks in the blockchain sequence.

SHA-256 may trigger block creation 416 may be connected to SHA-1 triggers user authentication 418. At SHA-1 triggers user authentication 418, SHA-1 algorithm functions to authenticate the user using a hash.

SHA-1 may trigger user authentication 418 may be connected to authentication engine 420. Authentication engine 420 may include authentication algorithms that may authenticate users and blockchains.

Authentication engine 420 may run an authentication pass? 422 algorithm over the blockchain produced. Authentication pass? 422 may determine whether the blockchain produced may be authenticated.

If the result of authentication pass? 422 is “No,” the blockchain may not be authenticated and the next step-send a number value to all other nodes as a passive block 424 may be performed. At send a number value to all other nodes as a passive block 424, a number value for a passive block is implemented at the other blockchain nodes. Then, the system may stop 426. At stop 426, block creation stops.

If the result of authentication pass? 422 is “Yes,” the blockchain may be authenticated. If the blockchain is authenticated, the system may proceed to verification with other nodes 428. At verification with other nodes 428, the blockchain may be verified by the other nodes and the requested transaction may proceed. Once verification with other nodes 428 is complete, the system may stop 426.

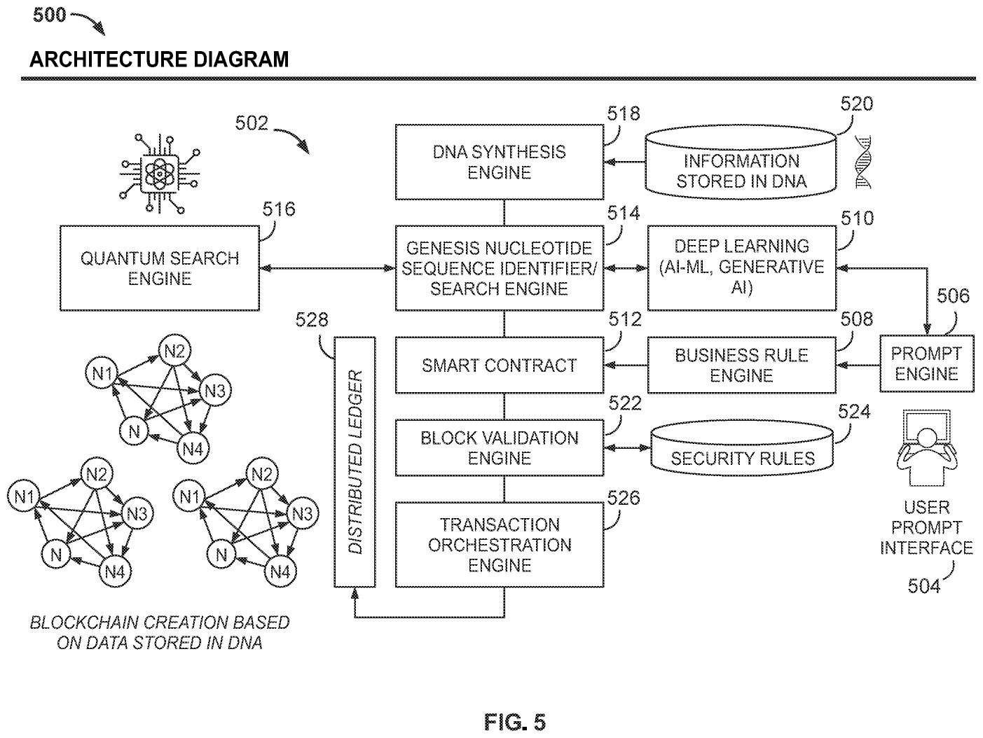

FIG. 5 shows an illustrative flow diagram 502 representing an architecture diagram 500 of the disclosure.

User prompt interface 504 may include a prompt engine 506. Prompt engine 506 may use generative AI and ML to analyze a transaction request for user requirements. Prompt engine 506 may be connected a business rule engine 508. Prompt engine 506 may be connected to deep learning 510. Deep learning 510 may contain AI-ML and generative AI.

Business rule engine 508 may include user requirements for a transaction request. Business rule engine 508 may be connected to smart contract 512. Smart contact 512 may produce smart contracts corresponding to user requirements for a transaction request.

Deep learning 510 may be connected to a genesis nucleotide sequence identifier and search engine 514. Genesis nucleotide sequence identifier and search engine 514 may include a nucleotide search engine and a genesis nucleotide search identifier. Genesis nucleotide sequence identifier and search engine 514 may be connected to a quantum search engine 516. Quantum search engine 516 may run the Grover's search algorithm.

Genesis nucleotide sequence identifier and search engine 514 may be connected to a DNA synthesis engine 518. DNA synthesis engine 518 may synthesize DNA based on results from the genesis nucleotide sequence identifier and search engine 514.

Based on the genesis nucleotide search identifier, the DNA synthesis engine 518 may synthesize a genesis nucleotide sequence. Based on the nucleotide search engine, the DNA synthesis engine 518 may synthesize other DNA nucleotide sequences. DNA synthesis engine 518 may be connected to information stored in DNA 520. DNA synthesis engine 518 may extract the information stored in DNA 520.

Genesis nucleotide sequence identifier and search engine 514 may be connected to smart contract 512. Smart contract 512 may produce smart contracts corresponding to user requirements. The smart contracts corresponding to user requirements may be converted to binary code. The binary code may be converted into DNA quaternary code (A-G-T-C). The DNA quaternary code may be read by the genesis nucleotide sequence identifier and search engine 514. Smart contract 512 may mint blocks on a blockchain based on the user requirements.

Smart contract 512 may be connected to a block validation engine 522. Block validation engine 522 may validate the blockchain minted by smart contract 512. Block validation engine 522 may be connected to security rules 524. Security rules 524 may contain a set of rules that control whether the blockchain is authenticated.

Block validation engine 522 may be connected to a transaction orchestration engine 526. Transaction orchestration engine 526 may orchestrate a transaction based on the validated blockchain from block validation engine 522.

The transaction orchestration engine 526 may place the blockchain on a distributed ledger 528. Distributed ledger 528 may include blockchain nodes, e.g., N, N1, N2, N3, and N4. Each blockchain node may be in network communication with one another.

The distributed ledger 528 may include multiple blockchains of transactions. Each blockchain of transactions may represent a separate transaction request. Thus, blockchain creation based on data stored in DNA is achieved.

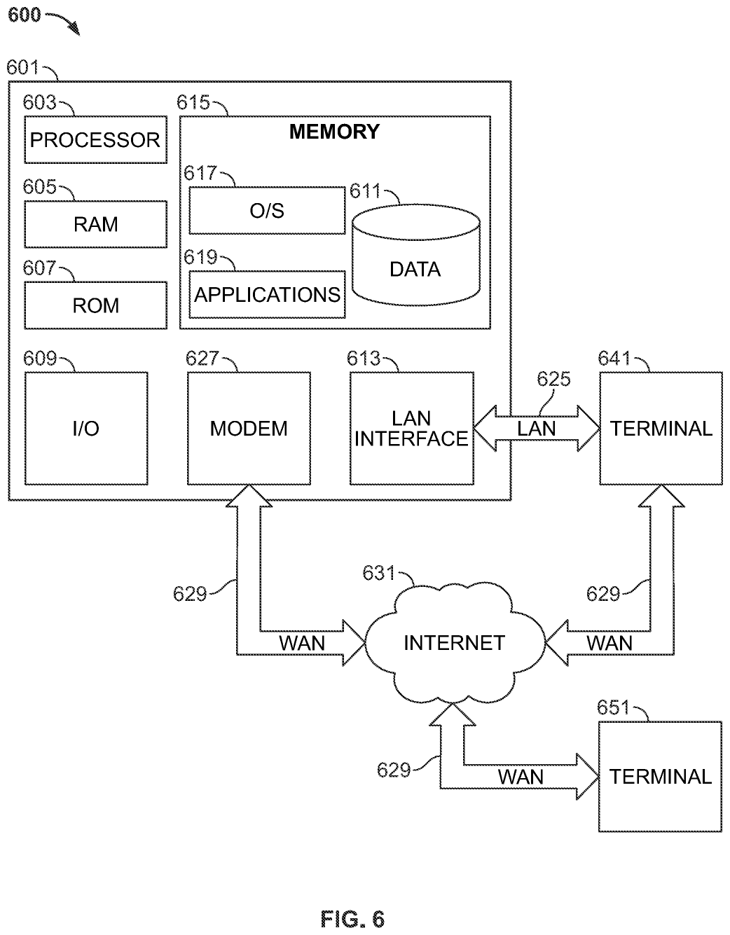

FIG. 6 shows an illustrative block diagram of system 600 that includes computer 601. Computer 601 may alternatively be referred to herein as a “server” or a “computing device.” Computer 601 may be a workstation, desktop, laptop, tablet, smart phone, ATM, satellite, or any other suitable computing device. Elements of system 600, including computer 601, may be used to implement various aspects of the systems and methods disclosed herein.

Computer 601 may have a processor 603 for controlling the operation of the device and its associated components, and may include RAM 605, ROM 607, input/output module 609, and memory 615. The processor 603 may also execute all software running on the computer—e.g., the operating system and/or voice recognition software. Other components commonly used for computers, such as EEPROM or Flash memory or any other suitable components, may also be part of the computer 601.

Memory 615 may be comprised of any suitable permanent storage technology—e.g., a hard drive. Memory 615 may store software including the operating system 617 and application(s) 619 along with any data 611 needed for the operation of the system 600. Memory 615 may also store videos, text, and/or audio assistance files. The videos, text, and/or audio assistance files may also be stored in cache memory, or any other suitable memory. Alternatively, some or all of computer executable instructions (alternatively referred to as “code”) may be embodied in hardware or firmware (not shown). Computer 601 may execute the instructions embodied by the software to perform various functions.

Input/output (“I/O”) module may include connectivity to a microphone, keyboard, touch screen, mouse, and/or stylus through which a user of computer 501 may provide input. The input may include input relating to cursor movement. The input may relate to database backup, search, and recovery. The input/output module may also include one or more speakers for providing audio output and a video display device for providing textual, audio, audiovisual, and/or graphical output. The input and output may be related to computer application functionality. The input and output may be related to database backup, search, and recovery.

System 600 may be connected to other systems via a local area network (“LAN”) interface 613. System 600 may operate in a networked environment supporting connections to one or more remote computers, such as terminals 641 and 651. Terminals 641 and 651 may be personal computers or servers that include many or all the elements described above relative to system 600.

The network connections depicted in FIG. 6 include a LAN 625 and a wide area network (“WAN”) 629 but may also include other networks. When used in a LAN networking environment, computer 601 is connected to LAN 625 through a LAN interface or adapter 613. When used in a WAN networking environment, computer 601 may include a modem 627 or other means for establishing communications over WAN 629, such as Internet 631.

It will be appreciated if the network connections shown are illustrative and other means of establishing a communications link between computers may be used. The existence of various well-known protocols such as TCP/IP, Ethernet, FTP, HTTP, and the like is presumed, and the system can be operated in a client-server configuration to permit a user to retrieve web pages from a web-based server. The web-based server may transmit data to any other suitable computer system. The web-based server may also send computer-readable instructions, together with the data, to any suitable computer system. The computer-readable instructions may be to store the data in cache memory, the hard drive, secondary memory, or any other suitable memory.

Additionally, application program(s) 619, which may be used by computer 601, may include computer executable instructions for invoking user functionality related to communication, such as e-mail, Short Message Service (“SMS”), and voice input and speech recognition applications. Application program(s) 619 (which may be alternatively referred to herein as “plugins,” “applications,” or “apps”) may include computer executable instructions for invoking user functionality related performing various tasks. The various tasks may be related to database backup, search, and recovery.

Computer 601 and/or terminals 641 and 651 may also be devices including various other components, such as a battery, speaker, and/or antennas (not shown).

Terminal 651 and/or terminal 641 may be portable devices such as a laptop, cell phone, Blackberry™, tablet, smartphone, or any other suitable device for receiving, storing, transmitting and/or displaying relevant information. Terminals 651 and/or terminal 641 may be other devices. These devices may be identical to system 600 or different. The differences may be related to hardware components and/or software components.

Any information described above in connection with database 611, and any other suitable information, may be stored in memory 615. One or more of applications 619 may include one or more algorithms that may be used to implement features of the disclosure, and/or any other suitable tasks.

The disclosure may be operational with numerous other general purpose or special purpose computing system environments or configurations. Examples of well-known computing systems, environments, and/or configurations that may be suitable for use with the disclosure include, but are not limited to, personal computers, server computers, hand-held or laptop devices, tablets, mobile phones, smart phones and/or other personal digital assistants (“PDAs”), multiprocessor systems, microprocessor-based systems, set top boxes, programmable consumer electronics, network PCs, minicomputers, mainframe computers, distributed computing environments that include any of the above systems or devices, and the like.

The disclosure may be described in the general context of computer-executable instructions, such as program modules, being executed by a computer. Generally, program modules include routines, programs, objects, components, data structures, etc., that perform tasks or implement abstract data types. The disclosure may also be practiced in distributed computing environments where tasks are performed by remote processing devices that are linked through a communications network. In a distributed computing environment, program modules may be in both local and remote computer storage media including memory storage devices.

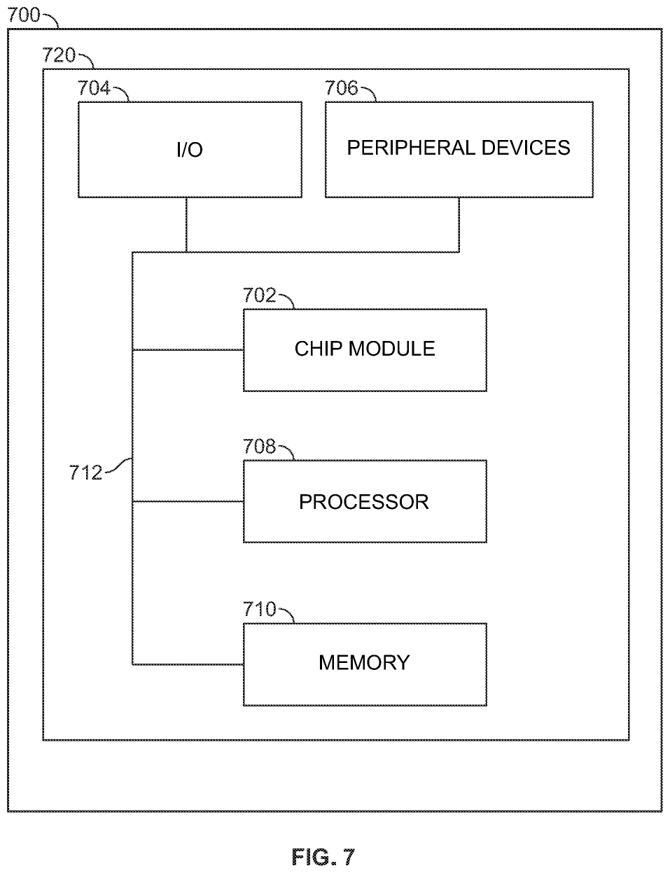

FIG. 7 shows illustrative system 700 that may be configured in accordance with the principles of the disclosure. System 700 may be a computing machine. System 700 may include one or more features of the system shown in FIG. 6. System 700 may include chip module 702, which may include one or more integrated circuits, and which may include logic configured to perform any other suitable logical operations.

System 700 may include one or more of the following components: I/O circuitry 704, which may include a transmitter device and a computer device and may interface with fiber optic cable, coaxial cable, telephone lines, wireless devices, PHY layer hardware, a camera/display control device or any other suitable media or devices; peripheral devices 706, which may include counter timers, real-time timers, power-on reset generators or any other suitable peripheral devices; logical processing device 708, which may compute data structural information and structural parameters of the data; and machine-readable memory 710.

Machine-readable memory 710 may be configured to store in machine-readable data structures: machine executable instructions (which may be alternatively referred to herein as “computer instructions” or “computer code”), applications, signals, and/or any other suitable information or data structures.

Components 702, 704, 706, 708 and 710 may be coupled together by a system bus or other interconnections 712 and may be present on one or more circuit boards such as 720. In some embodiments, the components may be integrated into a single chip. The chip may be silicon-based.

One of ordinary skill in the art will appreciate that the elements shown and described herein may be performed in other than the recited order and that one or more elements illustrated may be optional. The methods of the above-referenced embodiments may involve the use of any suitable elements, elements, computer-executable instructions, or computer-readable data structures. In this regard, other embodiments are disclosed herein as well that can be partially or wholly implemented on a computer-readable medium, for example, by storing computer-executable instructions or modules or by utilizing computer-readable data structures.

Thus, methods and systems for autonomously controlling and monitoring ATMs, leveraging a network of low-orbit satellites, during an ATM distress event, are provided. Persons skilled in the art will appreciate that the present invention can be practiced by other than the described embodiments, which are presented for purposes of illustration rather than of limitation, and that the present invention is limited only by the claims that follow.

What is claimed is:

1. A method for synthesizing a deoxyribonucleic acid (“DNA”) sequence and generating a distributed blockchain of transactions based on information stored in the DNA sequence leveraging artificial intelligence (“AI”), machine learning (“ML”), and a quantum search engine, the method comprising:
receiving a request to execute a transaction from a generative AI user prompt interface;
in response to receipt of the request:
analyzing the request to identify user requirements associated with the request;
generating a smart contract for each of the user requirements;
converting each of the user requirements to a binary code; and
synthesizing a DNA sequence including nucleotides, each group of the nucleotides corresponding to one of the binary codes, wherein the synthesizing encodes the user requirements associated with the request;
after the synthesizing of the DNA sequence, producing a blockchain storing data relating to the transaction using data stored in the DNA sequence, the producing comprising:
extracting data stored in the DNA sequence;
decoding the data extracted from the DNA sequence;
identifying, in the decoded data, one of the user requirements;
in response to identifying the user requirement, searching a database for a genesis nucleotide sequence corresponding to the user requirement, the searching being performed by each of AI-ML, generative AI, and a quantum search engine;
in response to identifying, in the database, the genesis nucleotide sequence, minting a genesis block, the genesis block storing the genesis nucleotide sequence;
initiating the blockchain of transactions by storing the genesis block on the blockchain;
minting a plurality of blocks to add to the blockchain, each of the plurality of blocks corresponding to each of the user requirements associated with the request;
after the minting of the genesis block and the plurality of blocks, adding the genesis block and the plurality of blocks to the blockchain and validating the blockchain using a blockchain validation engine;
searching the blockchain for a block termination nucleotide sequence via AI-ML, generative AI, and a quantum search engine;
identifying one of the plurality of blocks as the block termination nucleotide sequence; and
finalizing the blockchain by converting the identified one of the plurality of blocks to a terminal block on the blockchain, the terminal block storing the block termination nucleotide sequence.

2. The method of claim 1, further comprising, after the extracting of the data stored in the DNA sequence, mapping the data extracted to a contextual data interface using AI-ML, and mapping the contextual data interface further to the user requirements.

3. The method of claim 1, further comprising, after the minting of the genesis block, storing the genesis block on a network of computing systems, wherein each computing system is a node in the blockchain.

4. The method of claim 3, further comprising, after the minting of the plurality of blocks, storing the plurality of blocks on the network of computing systems.

5. The method of claim 1, wherein:
each group of the nucleotides in the DNA sequence corresponding to each one of the binary codes includes adenosine (“A”), thymine (“T”), cytosine (“C”), and guanine (“G”); and
A corresponds to “00;”
T corresponds to “01;”
C corresponds to “10;” and
G corresponds to “11.”

6. The method of claim 1, wherein the quantum search engine uses Grover's search algorithm to search for data stored in the DNA sequence.

7. The method of claim 6, wherein the quantum search engine enables faster searching of data required for the finalizing of the blockchain.

8. An apparatus for synthesizing a deoxyribonucleic acid (“DNA”) sequence and generating a distributed blockchain of transactions based on information stored in the DNA sequence leveraging artificial intelligence (“AI”), machine learning (“ML”), and a quantum search engine, the apparatus comprising:
a computer processor; and
a computer-readable medium, coupled with the computer processor, having instructions stored thereon, which, when executed by the computer processor, cause the computer processor to perform a method, the method comprising:
receiving a request to execute a transaction from a generative AI user prompt interface;
in response to receipt of the request:
analyzing the request to identify user requirements associated with the request;
generating a smart contract for each of the user requirements;
converting each of the user requirements to a binary code; and
synthesizing a DNA sequence including nucleotides, each group of the nucleotides corresponding to one of the binary codes, wherein the synthesizing encodes the user requirements associated with the request;
after the synthesizing of the DNA sequence, producing a blockchain storing data relating to the transaction using data stored in the DNA sequence, the producing comprising:
extracting data stored in the DNA sequence;
decoding the data extracted from the DNA sequence;
identifying, in the decoded data, one of the user requirements;
in response to identifying the user requirement, searching a database for a genesis nucleotide sequence corresponding to the user requirement, the searching being performed by each of AI-ML, generative AI, and a quantum search engine;
in response to identifying, in the database, the genesis nucleotide sequence, minting a genesis block, the genesis block storing the genesis nucleotide sequence;
initiating the blockchain of transactions by storing the genesis block on the blockchain;
minting a plurality of blocks to add to the blockchain, each of the plurality of blocks corresponding to each of the user requirements associated with the request;
after the minting of the genesis block and the plurality of blocks, adding the genesis block and the plurality of blocks to the blockchain and validating the blockchain using a blockchain validation engine;
searching the blockchain for a block termination nucleotide sequence via AI-ML, generative AI, and a quantum search engine;
identifying one of the plurality of blocks as the block termination nucleotide sequence; and
finalizing the blockchain by converting the identified one of the plurality of blocks to a terminal block on the blockchain, the terminal block storing the block termination nucleotide sequence.

9. The apparatus of claim 8, further comprising, after the extracting of the data stored in the DNA sequence, mapping the data extracted to a contextual data interface using AI-ML, and mapping the contextual data interface further to the user requirements.

10. The apparatus of claim 8, further comprising, after the minting of the genesis block, storing the genesis block on a network of computing systems, wherein each computing system is a node in the blockchain.

11. The apparatus of claim 10, further comprising, after the minting of the plurality of blocks, storing the plurality of blocks on the network of computing systems.

12. The apparatus of claim 8, wherein:
each group of the nucleotides in the DNA sequence corresponding to each one of the binary codes includes adenosine (“A”), thymine (“T”), cytosine (“C”), and guanine (“G”); and
A corresponds to “00;”
T corresponds to “01;”
C corresponds to “10;” and
G corresponds to “11.”

13. The apparatus of claim 8, wherein the quantum search engine uses Grover's search algorithm to search for data stored in the DNA sequence.

14. The apparatus of claim 13, wherein the quantum search engine enables faster searching of data required for the finalizing of the blockchain.

15. A system for synthesizing a deoxyribonucleic acid (“DNA”) sequence and generating a distributed blockchain of transactions based on information stored in the DNA sequence leveraging a quantum search engine, the system comprising:
a computer processor; and
a computer-readable medium, coupled with the computer processor, having instructions stored thereon, which, when executed by the computer processor, cause the computer processor to perform a method, the method comprising:
receiving a request to execute a transaction from a generative artificial intelligence (“AI”) user prompt interface;
in response to receipt of the request:
analyzing the request to identify user requirements associated with the request;
generating a smart contract for each of the user requirements;
converting each of the user requirements to a binary code; and
synthesizing a DNA sequence including nucleotides, each group of the nucleotides corresponding to one of the binary codes, wherein the synthesizing encodes the user requirements associated with the request;
after the synthesizing of the DNA sequence, producing a blockchain storing data relating to the transaction using data stored in the DNA sequence, the producing comprising:
extracting data stored in the DNA sequence;
decoding the data extracted from the DNA sequence;
identifying, in the decoded data, one of the user requirements;
in response to identifying the user requirement, searching a database for a genesis nucleotide sequence corresponding to the user requirement, the searching being performed by a quantum search engine;
in response to identifying, in the database, the genesis nucleotide sequence, minting a genesis block, the genesis block storing the genesis nucleotide sequence;
initiating the blockchain of transactions by storing the genesis block on the blockchain;
minting a plurality of blocks to add to the blockchain, each of the plurality of blocks corresponding to each of the user requirements associated with the request;
after the minting of the genesis block and the plurality of blocks, adding the genesis block and the plurality of blocks to the blockchain and validating the blockchain using a blockchain validation engine;
searching the blockchain for a block termination nucleotide sequence via a quantum search engine;
identifying one of the plurality of blocks as the block termination nucleotide sequence; and
finalizing the blockchain by converting the identified one of the plurality of blocks to a terminal block on the blockchain, the terminal block storing the block termination nucleotide sequence.

16. The system of claim 15, further comprising, after the extracting of the data stored in the DNA sequence, mapping the data extracted to a contextual data interface using AI-machine learning (“ML”), and mapping the contextual data interface further to the user requirements.

17. The system of claim 15, further comprising, after the minting of the genesis block, storing the genesis block on a network of computing systems, wherein each computing system is a node in the blockchain.

18. The system of claim 17, further comprising, after the minting of the plurality of blocks, storing the plurality of blocks on the network of computing systems.

19. The system of claim 15, wherein:
each group of the nucleotides in the DNA sequence corresponding to each one of the binary codes includes adenosine (“A”), thymine (“T”), cytosine (“C”), and guanine (“G”); and
A corresponds to “00;”
T corresponds to “01;”
C corresponds to “10;” and
G corresponds to “11.”

20. The system of claim 15, wherein the quantum search engine uses Grover's search algorithm to search for data stored in the DNA sequence.


SEQUENCE LISTING (1 sequence)

SEQ ID NO: 1 - DNA, 45 bp, unidentified
  source 1..45: mol_type = genomic DNA
gcagacgcccgtacgtacgttcaccgtgcgtcttcaccgtgcgtc